(19) United States
(12) Patent Application Publication
(10) Pub. No.: US 2014/0370027 A1
(43) Pub. Date: Dec. 18, 2014

(54) INHIBITION OF MACROPHAGE-SYNTHESIZED WNT7B TO INHIBIT TUMOR ANGIOGENESIS AND METASTASIS

(71) Applicants: Jeffrey W. Pollard, Edinburgh (GB); Richard A. Lang, Cincinnati, OH (US)

(72) Inventors: Jeffrey W. Pollard, Edinburgh (GB); Richard A. Lang, Cincinnati, OH (US)

(73) Assignees: Cincinnati Childrens's Hospital Medical Center, Cincinnati, OH (US); Albert Einstein College of Medicine of Yeshiva University, Bronx, NY (US)

(21) Appl. No.: 13/982,588

(22) PCT Filed: Mar. 30, 2012
(86) PCT No.: PCT/US12/31488

§ 371 Date: Oct. 11, 2013

Related U.S. Application Data
(60) Provisional application No. 61/470,740, filed on Apr. 1, 2011.

(51) Int. Cl. A61K 38/17; G01N 33/574; A61K 31/4709; C07K 16/18; C12N 15/113

(52) U.S. Cl. CPC A61K 38/177; C07K 16/18; C12N 15/113; A61K 31/4709; G01N 33/57484; C12N 2310/14; C12N 2310/531; G01N 2333/435; A61K 2039/505
USPC 424/156.1; 424/174.1; 514/44 A; 514/314; 514/19.8; 436/501

(57) ABSTRACT

The present invention provides methods of inhibiting angiogenesis in a malignant tumor, inhibiting metastasis of a malignant tumor and of inhibiting transformation of a benign tumor to a malignant tumor. The present invention also provides assays for identifying inhibitors of angiogenesis in a malignant tumor, inhibitors of metastasis of a malignant tumor and of inhibitors of transformation of a benign tumor to a malignant tumor.

### CROSS-REFERENCE TO RELATED APPLICATIONS

This application claims benefit of U.S. Provisional Application No. 61/470,740, filed Apr. 1, 2011, the contents of which are hereby incorporated by reference.

### STATEMENT OF GOVERNMENT SUPPORT

This invention was made with government support under grant number RO1CA131270 awarded by the National Institute on Aging, National Institutes of Health, U.S. Department of Health and Human Services. The government has certain rights in the invention.

### FIELD OF THE INVENTION

The present invention relates generally to inhibiting angiogenesis in tumors and inhibiting metastasis.

### BACKGROUND OF THE INVENTION

The disclosures of the references, patents, patent application publications and books referred to in the specification, and at the end of the specification, are hereby incorporated by reference in their entirety into the subject application to more fully describe the art to which the subject invention pertains.

The “.txt” Sequence Listing filed by EFS and which is entitled 96700_{—}1817_ST25.txt, is 11 kilobytes in size and which was created on Mar. 15, 2012 is hereby incorporated by reference.

Tumors require angiogenesis to support their growth. This vasculature increases dramatically at the benign to malignant transition, a process referred to as the angiogenic switch. This angiogenic switch is thought to be required for the malignant transition and for the activation of dormant metastases. These observations have resulted in the development of anti-angiogenic drugs, e.g. Avastin®, that are in clinical use in many cancers.

Anti-angiogenic anti-VEGF therapies are currently available. However, these are associated with several toxicities including unrestrained bleeding, gastrointestinal problems and death. Many cell types also make VEGF and thus targeting to the tumor alone is difficult.

Novel less harmful, anti-angiogenic and anti-metastatic therapies are needed. The present application addresses this need.

### SUMMARY OF THE INVENTION

A method of inhibiting formation of blood vessels in a malignant tumor in a subject, or of inhibiting metastasis of a malignant tumor in a subject, comprising administering to the subject an inhibitor of Wnt7b in an amount effective to inhibit formation of blood vessels in a malignant tumor or metastasis of a malignant tumor, respectively.

Also provided is a method of inhibiting a benign tumor from transforming into a malignant tumor in a subject comprising administering to the subject an inhibitor of Wnt7b in an amount effective to inhibit a benign tumor from transforming into a malignant tumor.

Also provided is a method for identifying an agent as an inhibitor of angiogenesis and/or vasculogenesis in a malignant tumor and/or as an inhibitor of metastasis of a malignant tumor, comprising:

contacting an amount of Wnt7b with an amount of the agent and determining whether the agent inhibits the activity of Wnt7b,
wherein if the agent inhibits the activity of Wnt7b then the agent is an inhibitor of angiogenesis and/or vasculogenesis in a malignant tumor or an inhibitor of metastasis of a malignant tumor, and wherein if the agent does not inhibit the activity of Wnt7b then the agent is not identified as an inhibitor of angiogenesis and/or vasculogenesis in a malignant tumor and/or an inhibitor of metastasis of a malignant tumor.

Also provided is a method for identifying an agent as an inhibitor of transformation of a benign tumor to a malignant tumor comprising: contacting an amount of Wnt7b with an amount of the agent and determining whether the agent inhibits the activity of Wnt7b,

wherein if the agent inhibits the activity of Wnt7b then the agent is an inhibitor of transformation of a benign tumor to a malignant tumor, and wherein if the agent does not inhibit the activity of Wnt7b then the agent is not identified as an inhibitor of transformation of a benign tumor to a malignant tumor.

Also provided is an agent which binds Wnt7b for treating a malignant tumor in a subject or for inhibiting metastasis in a subject or for preventing transformation of a benign tumor to a malignant tumor in a subject.

### BRIEF DESCRIPTION OF THE DRAWINGS

FIG. 1A-1J. Deletion of Wnt7b in tumor stroma cells results in reduced mammary tumor mass. Flow-sorting for isolation of human TAMs using the markers CD45, CD11 b, CD14 and CD163 was performed and then (1A) PCR amplification for WNT7B from mammary gland, ovarian tissue and endometrium. (1B) End-point RTPCR for Wnt7b mRNA in dextran+, F4/80+ macrophages, CD45+, CD3+ T and CD45+, B220+ B cells flow sorted from wild type MMTV-PyMT tumor. (1C) End-point RT-PCR for Wnt7b mRNA in flow-sorted dextran+, F4/80+ macrophages from Wnt7btm2Amc/− (control, C) and Wnt7btm2Amc/−; Csflr-icre (mutant, M) tumors. (1D) As in (B), but for CD45+, CD3+ T cells. (1E) As in (B), but for CD45+, B220+ B cells. (1F) Relative Wnt7b mRNA expression in control and mutant MMTVPyMT mammary tumors at premalignant (Hyperplasia and Adenoma, H/A), or malignant (early carcinoma, EC, and late carcinoma, LC) stages. (1G) Percentage of marker positive cells in control (MMTV-PyMT; Wnt7btm2Amc/−, C) and mutant (MMTV-PyMT; Wnt7bim2Amc/−; cfms-iCre, M) tumors combined from the 20-22 week range. (1H) Control and mutant inguinal gland volume from 6 weeks to 22 weeks. (1I, 1J) Inguinal gland mass at 16 (1I) and 22 (1J) weeks. For (1F-1J) sample number is shown at the base of the chart.

FIG. 2A-2F. Tumor progression and angiogenic switch are inhibited in the absence of Wnt7b in tumor stroma cells. (2A) Distribution of progression stage of PyMT mammary tumors at H/A lesions, EC and LC between genotypes and at the age of 16 weeks (for control, C, n=18, for mutant, M, n=18) and 22 weeks (for C, n=17, for M, n=9). (2B) Percentage of CD31+, CD105+ vascular endothelial cells in control and mutant tumors combined from the 20-22 week range. Blood vessels were perfused with TEXAS RED in PyMT mammary tumors at H/A lesions, EC and LC. (2C, 2E). Quantification of dextran labeled vessels for a given tumor stage using either vessels per field (2C) or branch-points per field (2E). (2D, 2F) Quantification of CD31 labeling in control and mutant late carcinomas shown either as CD31+ area (2D) or CD31+ vessels (2F). For (2B, 2C and 2D) sample number is shown at base of histogram bar.

FIG. 3A-3D. The expression of VEGFA is reduced by the deletion of tumor stroma cell Wnt7b. (3A) Relative VEGFA mRNA expression in control (3C) and mutant (M) MMTV-PyMT tumors at H/A, EC and LC stages. Inset: Immunoblotting for VEGFA and actin control and mutant MMTVPyMT tumor lysates. (3B) Relative sFlt-1 mRNA level in control and mutant MMTV-PyMT tumors at H/A, EC and LC stages. (3C, 3D) Relative expression of Axin2, c-Myc, and CyclinD1 in control and mutant whole tumor (3C) and CD31+, CD105+ blood vascular endothelial cells (3D). Sample number is shown at the base of each histogram bar.

FIG. 4A-4F. Metastasis is reduced in the absence of tumor stroma cell Wnt7b.

(4A-4F) Representative images of metastasized lung in control (4A, 4B) and mutant (4C) tumor at 22 weeks. Obvious surface metastases are marked with yellow dots. (4D) Quantification of total lung weight in control and mutant, tumor free and tumor bearing mice at 22 weeks. (4E) Relative PyMT mRNA expression in control and mutant lung at 22 weeks. (4F) The lung metastasis index for control and mutant mice at 22 weeks. Sample number is shown at the base of each histogram bar.

### DETAILED DESCRIPTION OF THE INVENTION

A method of inhibiting formation of blood vessels in a malignant tumor in a subject, and/or of inhibiting metastasis of a malignant tumor in a subject, comprising administering to the subject an inhibitor of Wnt7b in an amount effective to inhibit formation of blood vessels in the malignant tumor in the subject and/or metastasis of the malignant tumor in the subject, respectively.

In an embodiment, the inhibitor of Wnt7b is an inhibitor of macrophage-secreted Wnt7b. In an embodiment, the inhibitor of Wnt7b is an inhibitor of hematopoietic stroma Wnt7b. In an embodiment, the inhibitor of Wnt7b inhibits angiogenesis in the malignant tumor. In an embodiment, the inhibitor of Wnt7b inhibits vasculogenesis in the malignant tumor. In an embodiment, the inhibitor of Wnt7b inhibits metastasis of the malignant tumor.

In an embodiment, the malignant tumor is a tumor of the breast, prostate, lung, liver, pancreas, kidney, ovary, testicle, uterus, glia, central nervous system, oesphagus, stomach, colon, or is a glioblastoma, or wherein the metastasis is a pulmonary, bone, liver, breast, melanoma, kidney, cervix, or colon metastasis.

In an embodiment, the inhibitor of Wnt7b is an antibody which binds Wnt7b or a fragment of an antibody which fragment binds Wnt7b. In an embodiment, the inhibitor of Wnt7b is a monoclonal antibody or a fragment of a monoclonal antibody. In an embodiment, the inhibitor of Wnt7b is an aptamer. In an embodiment, the inhibitor of Wnt7b is an antisense which inhibits expression of Wnt7b. In an embodiment, the inhibitor of Wnt7b is an siRNA which inhibits expression of Wnt7b or an shRNA which inhibits expression of Wnt7b. In an embodiment, the inhibitor of Wnt7b is a small organic molecule of 2000 daltons or less. In an embodiment, the small organic molecule is pyrvinium (2-[(E)-2-(2,5-Dimethyl-1-phenylpyrrol-3-yl)ethenyl]-N,N,1-trimethylquinolin-1-ium-6-amine). In an embodiment, the inhibitor is a secreted frizzled protein or active fragment thereof.

A method of inhibiting a benign tumor from transforming into a malignant tumor in a subject comprising administering to the subject an inhibitor of Wnt7b in an amount effective to inhibit the benign tumor from transforming into a malignant tumor.

In an embodiment, inhibitor of Wnt7b is an inhibitor of macrophage-secreted Wnt7b. In an embodiment, the benign tumor is a benign tumor of the breast, prostate, lung, liver, pancreas, kidney, ovary, testicle, uterus, glia, central nervous system, oesphagus, stomach, colon, or glia. In an embodiment, the inhibitor of Wnt7b is an antibody which binds Wnt7b or a fragment of an antibody which fragment binds Wnt7b. In an embodiment, the inhibitor of Wnt7b is a monoclonal antibody or a fragment of a monoclonal antibody. In an embodiment, the inhibitor of Wnt7b is an aptamer. In an embodiment, the inhibitor of Wnt7b is an antisense which inhibits expression of Wnt7b. In an embodiment, the inhibitor of Wnt7b is an siRNA which inhibits expression of Wnt7b or an shRNA which inhibits expression of Wnt7b. In an embodiment, the inhibitor of Wnt7b is a small organic molecule of 2000 daltons or less or of 1000 Daltons or less, or of 800 Daltons or less. In an embodiment, the small organic molecule is pyrvinium (2-[(E)-2-(2,5-Dimethyl-1-phenylpyrrol-3-yl)ethenyl]-N,N,1-trimethylquinolin-1-ium-6-amine). In an embodiment, the inhibitor is a secreted frizzled protein or an active fragment thereof.

In an embodiment, Wnt7b is inhibited in the hematopoietic stroma. In an embodiment, lung or lymph node metastasis is inhibited. In an embodiment, the method reduces vascular density in the tumor.

A method for identifying an agent as a candidate inhibitor of angiogenesis and/or vasculogenesis in a malignant tumor and/or as an inhibitor of metastasis of a malignant tumor, comprising:

contacting an amount of Wnt7b with an amount of the agent and determining whether the agent inhibits the Wnt7b,
wherein if the agent inhibits the Wnt7b then the agent is a candidate inhibitor of angiogenesis and/or vasculogenesis in a malignant tumor or a candidate inhibitor of metastasis of a malignant tumor, and wherein if the agent does not inhibit the Wnt7b then the agent is not identified as a candidate inhibitor of angiogenesis and/or vasculogenesis in a malignant tumor and/or a candidate inhibitor of metastasis of a malignant tumor.

In an embodiment, the method further comprises determining if the candidate inhibitor so identified is an inhibitor of angiogenesis and/or vasculogenesis in a malignant tumor and/or a candidate inhibitor of metastasis of a malignant tumor comprising administering the candidate inhibitor to a mammal having a malignant tumor and measuring angiogenesis and/or vasculogenesis in the malignant tumor or metastasis of the malignant tumor in the presence of the candidate inhibitor as compared to in the absence of the candidate inhibitor, wherein lower angiogenesis and/or vasculogenesis in the malignant tumor or less metastasis of the malignant tumor in the presence of the candidate inhibitor indicates that it is an inhibitor.

A method for identifying an agent as a candidate inhibitor of transformation of a benign tumor to a malignant tumor comprising:

contacting an amount of Wnt7b with an amount of the agent and determining whether the agent inhibits the Wnt7b,
wherein if the agent inhibits the Wnt7b then the agent is a candidate inhibitor of transformation of a benign tumor to a malignant tumor, and wherein if the agent does not inhibit the Wnt7b then the agent is not identified as a candidate inhibitor of transformation of a benign tumor to a malignant tumor.

In an embodiment, the method further comprises determining if the candidate inhibitor so identified is an inhibitor of transformation of a benign tumor to a malignant tumor comprising administering the candidate inhibitor to a plurality of subjects having a benign tumor and determining if in the presence of the candidate inhibitor as compared to in the absence of the candidate inhibitor, the transformation of a benign tumor to a malignant tumor occurs at a lower rate in the a plurality of subjects, wherein if a lower rate is observed in the presence then the candidate inhibitor is an inhibitor of transformation of a benign tumor to a malignant tumor.

The expression levels of Wnt7b can be determined by methods well known for determining protein levels such as immunoassays. The inhibition of Wnt7b can be determined as inhibition of the angiogenic and/or vasculogenic properties of Wnt7b.

In an embodiment, the agent is an antibody, a fragment of an antibody, an aptamer, an siRNA, an shRNA, an antisense or a small organic molecule of 2000 daltons or less or of 1000 Daltons or less, or of 800 Daltons or less, a secreted frizzled protein or a fragment thereof.

In an embodiment of the methods, the Wnt7b is human Wnt7b comprising the sequence set forth in SEQ ID NO:1.

In an embodiment of the methods, the subject is a mammal. In an embodiment of the treatment methods, the subject is a human.

An agent which binds Wnt7b for treating a malignant tumor in a subject or for inhibiting metastasis in a subject or for preventing transformation of a benign tumor to a malignant tumor in a subject.

In an embodiment the agent is an antibody which binds Wnt7b or a fragment of an antibody which fragment binds Wnt7b. In an embodiment the antibody is a monoclonal antibody or the fragment of the antibody is a fragment of a monoclonal antibody. In an embodiment the agent is an aptamer, an siRNA, an shRNA, an antisense or a small organic molecule of 2000 daltons or less or of 1000 Daltons or less, or of 800 Daltons or less. In an embodiment the Wnt7b is human Wnt7b having the sequence set forth in SEQ ID NO:1.

As used herein a “malignant tumor” is a detectable malignant tumor usually presenting as a lesion or lump located in an organ or tissue in a subject, and may also be present in adjacent organs and or tissues in a subject. A “benign tumor” as used herein is a tumor that has not progressed to a malignant state.

As used herein “metastasize” means, in regard to a cancer or tumor, to spread from one organ or tissue of a subject to another non-adjacent organ or tissue of the subject.

In an embodiment, the Wnt7b is human Wnt7b protein (e.g. NP_{—}478679). In an embodiment the Wnt7b has the sequence:

(SEQ ID NO: 1)

| | |
|---|---|
| 1 | MHRNFRKWIF YVFLCFGVLY VKLGALSSVV ALGANIICNK |
| | IPGLAPRQRA ICQSRPDAII |
| 61 | VIGEGAQMGI NECQYQFRFG RWNCSALGEK TVFGQELRVG |
| | SREAAFTYAI TAAGVAHAVT |
| 121 | AACSQGNLSN CGCDREKQGY YNQAEGWKWG GCSADVRYGI |
| | DFSRRFVDAR EIKKNARRLM |
| 181 | NLHNNEAGRK VLEDRMQLEC KCHGVSGSCT TKTCWTTLPK |
| | FREVGHLLKE KYNAAVQVEV |
| 241 | VRASRLRQPT FLRIKQLRSY QKPMETDLVY IEKSPNYCEE |
| | DAATGSVGTQ GRLCNRTSPG |
| 301 | ADGCDTMCCG RGYNTHQYTK VWQCNCKFHW CCFVKCNTCS |
| | ERTEVFTCK |

As used herein, a shRNA (small hairpin RNA) or siRNA (small interfering RNA) “directed to” a target means an shRNA or siRNA, respectively, effective to inhibit expression of the target, in this case Wnt7b. In an embodiment, the siRNA as used in the methods or compositions described herein comprises a portion which is complementary to the mRNA encoding Wnt7b set forth in SEQ ID NO:2, and the siRNA is effective to inhibit expression of Wnt7b. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment the siRNA can be administered such that it is transfected into one or more cells.

In one embodiment, a siRNA of the invention comprises a double-stranded RNA wherein one strand of the double-stranded RNA is 80, 85, 90, 95 or 100% complementary to a portion of an RNA transcript of a gene encoding Wnt7b. In another embodiment, a siRNA of the invention comprises a double-stranded RNA wherein one strand of the RNA comprises a portion having a sequence the same as a portion of 18-25 consecutive nucleotides of an RNA transcript of a gene encoding Wnt7b. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In an embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In an embodiment, an siRNA of the invention comprises at least one 2′-sugar modification. In another embodiment, an siRNA of the invention comprises at least one nucleic acid base modification. In an embodiment, an siRNA of the invention comprises at least one phosphate backbone modification.

In an embodiment, the nucleic acid encoding Wnt7b has the sequence:

(SEQ ID NO: 2)

| | |
|---|---|
| 1 | ggatcatgca cagaaacttt cgcaagtgga |
| | ttttctacgt gtttctctgc tttggcgtcc |
| 61 | tgtacgtgaa gctcggagca ctgtcatccg |
| | tggtggccct gggagccaac atcatctgca |
| 121 | acaagattcc tggcctagcc ccgcggcagc |
| | gtgccatctg ccagagtcgg cccgatgcca |
| 181 | tcattgtgat tggggagggg gcgcagatgg |
| | gcatcaacga gtgccagtac cagttccgct |
| 241 | tcggacgctg gaactgctct gccctcggcg |
| | agaagaccgt cttcgggcaa gagctccgag |
| 301 | tagggagccg tgaggctgcc ttcacgtacg |
| | ccatcaccgc ggctggcgtg gcgcacgccg |
| 361 | tcaccgctgc ctgcagccaa gggaacctga |
| | gcaactgcgg ctgcgaccgc gagaagcagg |
| 421 | gctactacaa ccaagccgag ggctggaagt |
| | ggggcggctg ctcggccgac gtgcgttacg |
| 481 | gcatcgactt ctcccggcgc ttcgtggacg |
| | ctcgggagat caagaagaac gcgcggcgcc |
| 541 | tcatgaacct gcataacaat gaggccggca |
| | ggaaggttct agaggaccgg atgcagctgg |
| 601 | agtgcaagtg ccacggcgtg tctggctcct |
| | gcaccaccaa aacctgctgg accacgctgc |
| 661 | ccaagttccg agaggtgggc cacctgctga |
| | aggagaagta caacgcggcc gtgcaggtgg |
| 721 | aggtggtgcg ggccagccgt ctgcggcagc |
| | ccaccttcct gcgcatcaaa cagctgcgca |
| 781 | gctatcagaa gcccatggag acagacctgg |
| | tgtacattga gaagtcgccc aactactgcg |
| 841 | aggaggacgc ggccacgggc agcgtgggca |
| | cgcagggccg tctctgcaac cgcacgtcgc |
| 901 | ccggcgcgga cggctgtgac accatgtgct |
| | gcggccgagg ctacaacacc caccagtaca |
| 961 | ccaaggtgtg gcagtgcaac tgcaaattcc |
| | actggtgctg cttcgtcaag tgcaacacct |
| 1021 | gcagcgagcg caccgaggtc ttcacctgca |
| | agtgaggcca ggcccggagg cggccgcggg |
| 1081 | caccctggaa cccggcggca ttttgcacat |
| | ccactcctca ccttccctgc cttggtgctg |
| 1141 | ccagcagcag acatagacgg gtgcagaagc |
| | ggggagctcc aggtgcagga gggcaccggc |
| 1201 | cggggcccac gccctctgcc cgcctccctg |
| | gggctccttc ctgccacctc ctcccatcac |
| 1261 | ctcctgcggc agaacagcac ccgtgaccca |
| | cccagagagc aaggccaggg gtcttggtgc |
| 1321 | tccccgacgg ggcccggcaa gttctctttc |
| | ttctctctgg gaaaatgaac gtccaggaca |
| 1381 | cacctgtatc ccagagagca aagtgatgag |
| | gagactgagc gtccccagcc ccacctggcg |
| 1441 | gcatggacac agaaaagcta cgccggctgg |
| | cctctccaga ccagttccca ggctgggtct |
| 1501 | gctgggc cctggggcgg tggggacaga |
| | tgttgacaca aattatttat gttttcttag |
| 1561 | tatcagaaga ggattctcgg cactaacaca |
| | tagccagtcc taactccgta ctctgtgtca |
| 1621 | gcccatcccc tagacaccct ctgtttcctt |
| | tcccggcccc acctggccgg ccctctgccc |
| 1681 | ctgcagagct gaggcagcct ggggttgatg |
| | gggaccacgc ggtgcctgca ggtcctagaa |
| 1741 | gtgagctggg caggggctct tcagaccaca |
| | cagccctgac cgggccttgg aggagagcca |
| 1801 | tggacaggct cctccatgcc gtctttcctt |
| | cttttgaaaa tcctatcaat ggctgggcgc |
| 1861 | ggtggctcac acctgtaatc ccagcacttt |
| | gggagaccga ggcaggtgga tcacctgagg |
| 1921 | tcaggagttc gagaccagcc tggccaacgt |
| | ggtgaaaccc tgtctctact aaaaatacaa |
| 1981 | aaattagctg ggcgtggtgg cgtgcacctg |
| | taatcccagc tactcaggag gctgagacag |
| 2041 | gacacttgct tgaacccggg aggtggaggt |
| | tgcaatgagc caagattgtg ccactgtatt |
| 2101 | ccaacttggg tgacagagca cgactctgtc |
| | tcaaaaaaaa aaaaaaaaaa aaaaaaaaaa |

In an embodiment the nucleic acid is the gene WNT7b (NM_{—}058238.2). In an embodiment each occurrence of “t” in SEQ ID NO:2 is replaced with a “u” (i.e. uracil).

As used herein an “aptamer” is a single-stranded oligonucleotide or oligonucleotide analog that binds to a particular target molecule, such as a Wnt7b, or to a nucleic acid encoding a Wnt7b, and inhibits the function or expression thereof, as appropriate. Alternatively, an aptamer may be a protein aptamer which consists of a variable peptide loop attached at both ends to a protein scaffold that interferes with Wnt7b protein interactions.

As used herein an “antisense” is an antisense oligonucleotide, of between 5 and 45 nucleotide residues in length, directed to mRNA encoding Wnt7b.

As used herein, the term “antibody” refers to complete, intact antibodies, and “fragment” of an antibody refers to Fab, Fab′, F(ab)₂, and other fragments which fragments bind the antigen of interest, in this case Wnt7b. Complete, intact antibodies include, but are not limited to, monoclonal antibodies such as murine monoclonal antibodies, polyclonal antibodies, chimeric antibodies, human antibodies, and humanized antibodies. Fragments may be of any of the antibodies.

Various forms of antibodies may be produced using standard recombinant DNA techniques (Winter and Milstein, Nature 349: 293-99, 1991). For example, “chimeric” antibodies may be constructed, in which the antigen-binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments. In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly.

Another embodiment of the antibodies employed in the compositions and methods of the invention is a human antibody which binds human Wnt7b, which can be produced in nonhuman animals, such as transgenic animals harboring one or more human immunoglobulin transgenes. Such animals may be used as a source for splenocytes for producing hybridomas, as is described in U.S. Pat. No. 5,569,825.

Antibody fragments and univalent antibodies which bind human Wnt7b may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of specifically reacting with Wnt7b.

The antibody (intact of fragment) can be conjugated to a molecule which permits the antibody to cross the cell membrane or which aids in internalization of the antibody by adipocytes.

As used herein, the term “bind”, or grammatical equivalent, means the physical or chemical interaction between two proteins or compounds or associated proteins or compounds or combinations thereof, including the interaction between an antibody and a protein. Binding includes ionic, non-ionic, hydrogen bonds, Van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates.

The inhibitors of Wnt7b of this invention as used in the methods of this invention, may be administered in various forms, including those detailed herein. The treatment with the inhibitor of Wnt7b may be a component of a combination therapy or an adjunct therapy, i.e. the subject or patient in need of the drug is treated or given another drug for the disease (e.g. a chemotherapeutic) in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the recited inhibitors administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the inhibitors may comprise a single compound or mixtures thereof with anti-tumor and/anti-metastatic agents can be administered in oral dosage forms as tablets, capsules, pills, powders, granules, elixirs, tinctures, suspensions, syrups, and emulsions. The inhibitors may also be administered in intravenous (bolus or infusion), intraperitoneal, subcutaneous, or intramuscular form, or introduced directly, e.g. by injection or other methods, into the tumor, all using dosage forms well known to those of ordinary skill in the pharmaceutical arts.

The inhibitors can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The inhibitors can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be coadministered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36, James McGinity, Ed., 1989); Pharmaceutical Particulate Carriers: Therapeutic Applications: Drugs and the Pharmaceutical Sciences, Vol. 61 (Main Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The inhibitors can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The inhibitors may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The inhibitors can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field, the content of which is hereby incorporated by reference.

The inhibitor of Wnt7b of the instant invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

In an embodiment of the methods disclosed herein the subject is a human. In an embodiment of the methods disclosed herein the subject is woman. In an embodiment of the methods disclosed herein the subject is a man.

Where a numerical range is provided herein, it is understood that all numerical subsets of that range, and all the individual integers contained therein, are provided as part of the invention. Thus, an siRNA which is from 20 to 25 nucleotides in length includes the subset of siRNA which are 20 to 23 nucleotides in length, the subset of siRNA which are 22 to 24 nucleotides in length etc. as well as an siRNA which is 20 nucleotides in length, an siRNA which is 21 nucleotides in length, an siRNA which is 22 nucleotides in length, etc. up to and including an siRNA which is 25 nucleotides in length.

As used herein, “inhibiting” angiogenesis, or any grammatical equivalent thereof, means to reduce the amount of, or rate of, angiogenesis or preventing new angiogenesis.

As used herein, “inhibiting” vasculogenesis, or any grammatical equivalent thereof, means to reduce the amount of, or rate of, vasculogenesis or preventing vasculogenesis.

As used herein, “inhibiting” new blood vessel formation, or any grammatical equivalent thereof, means to reduce the amount of, or rate of, new blood vessel formation or preventing new blood vessel formation.

As used herein, “inhibiting” metastasis, or any grammatical equivalent thereof, means to reduce the extent of, or rate of, metastasis or to prevent metastasis.

As used herein to inhibit Wnt7b means to inhibit the angiogenic and/or vasculogenic activity of Wnt7b protein or means to inhibit expression of Wnt7b protein. Thus, an “inhibitor” of Wnt7b as used herein means an agent which inhibits the activity of, or expression, of Wnt7b. In an embodiment, the inhibitor inhibits the expression of Wnt7b. In an embodiment, the inhibitor inhibits the activity of Wnt7b. All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example 1

### Introduction

It has been well established that angiogenesis is required for the progression of tumors. Tumors require angiogenesis to support their growth. This vasculature increases dramatically at the benign to malignant transition, a process referred to as the angiogenic switch in which an obligate increase in vascular density occurs (Hanahan and Folkman, 1996). These observations have resulted in the development of anti-angiogenic drugs, e.g. Avastin®, that are in clinical use in many cancers. The most prominent therapy are anti-VEGF therapies that are used in a wide range of cancers. However, these therapies have significant side effects limiting their use.

The mechanism of controlling angiogenesis in tumors is still poorly understood. Genetic experiments in the present laboratory originally showed that depletion of macrophages blocked the angiogenic switch in autochthonous mammary tumors caused by expression of the Polyoma middle T oncoprotein (PyMT). Subsequent groups have confirmed these data. This laboratory and others have demonstrated in mouse models of breast cancer that tumor associated macrophages (TAMs) regulate the angiogenic switch and their depletion blocks this switch (De Palma et al., 2005; Lin et al., 2006). Transcriptional processing of unique populations of macrophages has indicated an enrichment of Wnt signaling molecules synthesized by TAMs (Ojalvo et al., 2010). Of these Wnt7b was of particular interest as it has been shown to be involved in vascular remodeling in the eye during development (Lobov et al., 2005).

Results

Herein is disclosed a novel mechanism for the regulation of this angiogenic switch that is affected through the macrophage-specific expression of Wnt7b. Thus, targeted depletion of Wnt7b in macrophages using mice carrying a floxed allele of Wnt7b crossed with macrophage-restricted cre-recombinase mouse strain completely blocks the angiogenic switch in the PyMT model. In addition, this loss of Wnt7b also inhibited pulmonary metastases. Wnt7b in human tumors is related to poor prognosis in human cancers (Ojalvo et al 2009). Inhibition of Wnt7b will inhibit tumor angiogenesis and metastasis. Wnts are use mostly during development and thus inhibition of Wnt7b in tumors is unlikely to cause significant toxicity in adults and thus provides novel anti-angiogenic therapies superior to those currently available.

Wnt7b was targeted herein using a macrophage-restricted cre developed in the Pollard lab (Csflr.icre) crossed with the PyMT model. Successful deletion of Wnt7b by the cre was achieved and reduction of Wnt7b mRNA in a PyMT tumor resulted. The inhibitory effect of Wnt7b depletion on tumor growth and progression was observed as was the inhibitory effect of Wnt7b depletion on the tumor vasculature visually (light color marks vessels). Depletion of macrophage-derived Wnt7b blocks the angiogenic switch at the hyperplasia/adenoma (H/A) to early carcinoma (EC) and this effect persists to late carcinoma (LC), and this was seen to extend to vessel integrity. The effect on vessel density was confirmed using anti-CD31 staining. A reduction in VEGF was observed following Wnt7b depletion suggesting that VEGF is downstream of Wnt7b signaling. Depletion of macrophage-derived Wnt7b also reduced metastasis as observed visually and as quantitated.

Thus, genetic deletion of macrophage-synthesized Wnt7b blocks the angiogenic switch and inhibits metastasis in mouse models of breast cancer caused by the mammary epithelial expression of PyMT.

### Example 2

### Introduction

Emerging evidence has shown that in mammary carcinoma, hematopoietic cells recruited to the tumor stroma contribute to tumor progression and metastasis. B lymphocytes contribute to tumorigenesis by enhancing chronic inflammation (Andreu et al., 2010; de Visser et al., 2005). A higher number of regulatory T cells is associated with higher grade tumors (Bailey et al., 2006) while CD4+ T cell depletion inhibits tumor recurrence (Matsui et al., 1999). Moreover, CD4+ T cells regulate tumor growth and pulmonary metastasis by promoting the pro-tumor activity of tumor-associated macrophages through IL-4 or IL-17 (DeNardo et al., 2009; Wang et al., 2009). Finally, extensive evidence shows that tumor-associated macrophages (TAMs) contribute to tumor progression (Lin et al., 2001) by remodeling extracellular matrix, stimulating angiogenesis, and promoting tumor cell intravasation and extravasation (Condeelis and Pollard, 2006; Qian et al., 2009).

The Wnt/β-catenin pathway has a critical role in normal development and tumorigenesis (Logan and Nusse, 2004). Low membrane β-catenin expression, its nuclear localization or stabilizing exon 3 mutations are significantly associated with poor cancer prognosis (Garcia-Rostan et al., 2001; Pukkila et al., 2001). Abnormal expression of APC, a negative regulator of β-catenin, is a major cause of colon cancer (Kinzler et al., 1991; Kinzler and Vogelstein, 1996; Korinek et al., 1997). Furthermore, the gene for Axin, another negative regulator of the Wnt/β-catenin pathway, is deleted in many types of cancer (Dahmen et al., 2001; Taniguchi et al., 2002). The notion that Wnt ligands may be involved in mammary tumor progression comes from mouse mammary tumor virus (MMTV) insertional activation of the Wnt1 gene (Nusse and Varmus, 1982), the observation that the ligands are expressed in human cancers (Bui et al., 1997; Huguet et al., 1994) and the demonstration that Wnt co-receptor Lrp5-deficient mice are resistant to Wnt1-induced mammary tumors (Lindvall et al., 2006). Recently, it has been reported that invasive TAMs from the MMTV-PyMT mouse model of mammary carcinoma secrete several Wnts, especially Wnt5b and Wnt7b (Ojalvo et al., 2010).

### Results

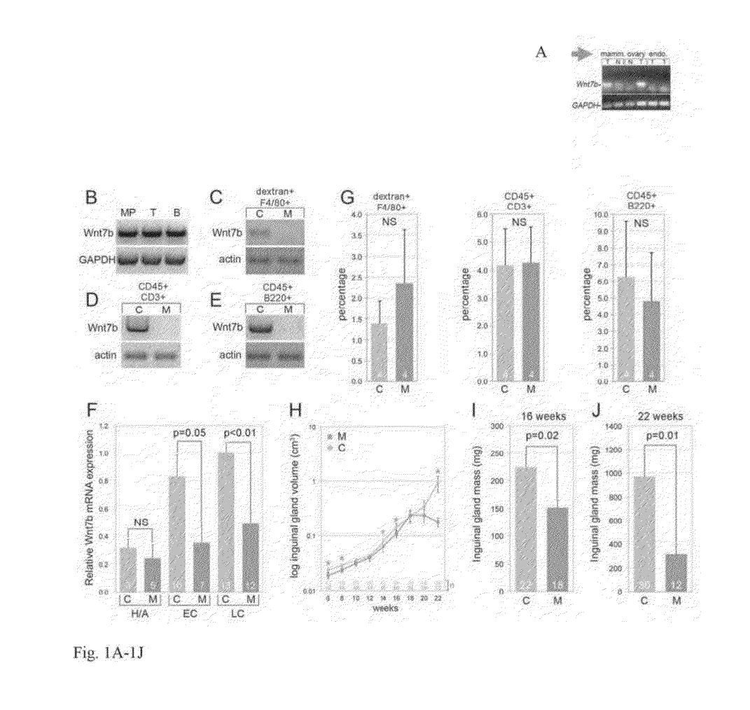

The Wnt family ligand Wnt7b has been implicated in modulation of vascularity (Lobov et al., 2005; Stenman et al., 2008) and in the function of tumor-promoting TAMs (Ojalvo et al., 2009a; Ojalvo et al., 2009b). To determine whether TAMs from human tumors expressed Wnt7b, we performed flow sorting of CD45+, CD11b+, CD14+, CD163+ myeloid cells from fresh primary tumor samples was performed and end-point PCR or Wnt7b (FIG. 1A). This analysis was performed on mammary, ovarian and endometrial tumors and showed that TAMs from both mammary and ovarian tumors showed strong amplification products for WNT7B (the gene) (FIG. 1A). Two samples of TAMs from endometrial tumors and normal ovarian tissue showed no amplification of WNT7B. Normal mammary tissue that was tumor-adjacent showed only a modest amplification of WNT7B. These data indicate that human TAMs from two different tumor types express WNT7B.

To assess the role of Wnt7b in the hematopoietic tumor stroma that includes TAMs, we generated conditional loss of function mice using the Wnt7b^{tm2Amc} allele (Lin et al., 2010) and the Csflr-icre transgene (Deng et al., 2010). End-point RT-PCR on flow sorted populations of F4/80+ macrophages, CD3+ T cells, and B220+ B cells from the tumor showed that these cell types all express Wnt7b (FIG. 1B) and that expression is undetectable in Wnt7b^{-/tm2Amc}; Csf1ricre mice (FIG. 1C-E). QPCR revealed that in the normal progression from hyperplasia/adenoma to early and late carcinoma, the expression level of Wnt7b increases in the MMTV-PyMT tumor (FIG. 1F, gray bar). By contrast, tumors from Wnt7b^{-/tm2Amc}; Csflr-icre mice show a level of Wnt7b expression that does not increase significantly with tumor progression (FIG. 1F, blue bars). This indicates that the hematopoietic tumor stroma is a major source of this progression associated Wnt ligand.

Deletion of Wnt7b from the hematopoietic tumor stroma did not significantly change the populations of recruited F4/80+ macrophages, CD3+ T cells, and B220+ B cells (FIG. 1G). To determine if Wnt7b from the hematopoietic stroma influenced tumor growth, gland volumes were recorded over the 6-22 week progression (FIG. 1H). Plotting these data on a log scale showed that though there were statistically significant differences at some time points between 6 and 18 weeks, in absolute terms, the differences were small and the growth curves nearly coincident (FIG. 1H). By contrast, there was a major divergence starting at 18 weeks in which control tumors increased in volume dramatically (FIG. 1H, gray trace) while the Wnt7b-deficient tumors decreased slightly in volume (FIG. 1H, blue trace). In the control group, the tumor volume at 22 weeks was 650% of the 16 week tumor volume. In the mutant group, 22 week tumor volume was 170% of the 16 week tumor volume. Tumor mass was also reduced in Wnt7b conditional mice. Tumors from the inguinal mammary glands were removed at 16 and 22 weeks and weighed. This showed that at both time-points, tumor mass in the mutant mice was significantly reduced compared with controls (FIG. 1I, J). At 22 weeks, tumor weight in the mutant mice was 30% of tumor weight in the control mice. These data show that Wnt7b from the hematopoietic stroma promotes tumor growth.

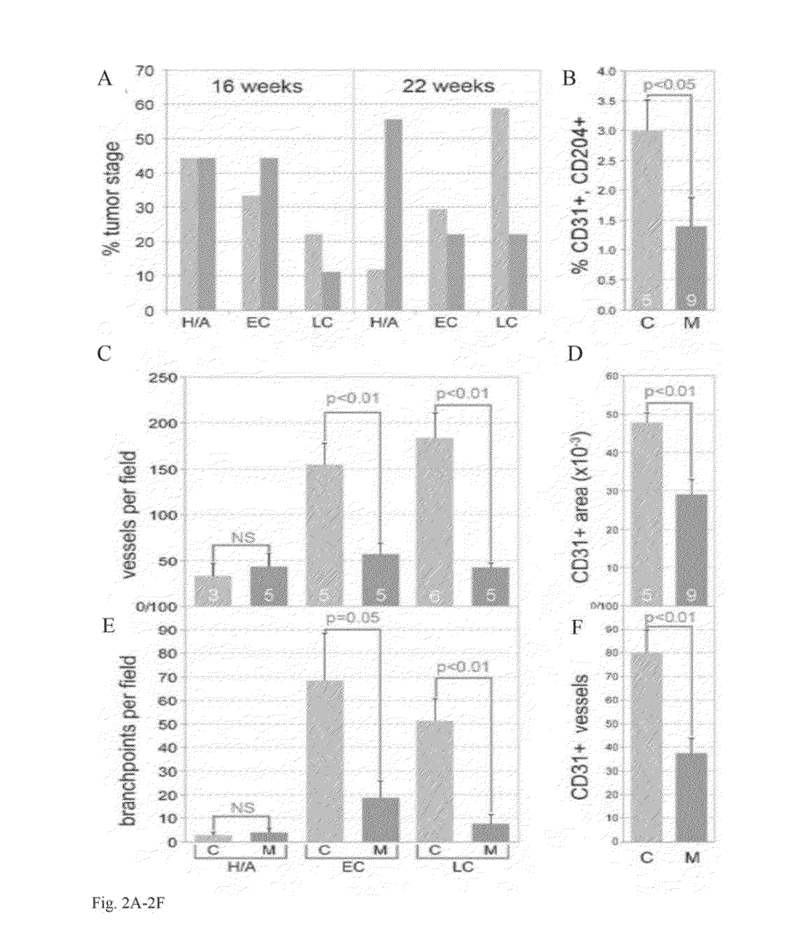

Prompted by the changes in tumor growth in the conditional mutant, it was then determined whether the stage of tumor progression was affected by Wnt7b deletion from the hematopoietic stroma. Histological tumor progression was assessed on the basis of representative sections from 4 planes in each inguinal mammary gland at 16 and 22 weeks. This analysis was performed blind using criteria described previously (Lin et al., 2003). At 16 weeks, control and mutant tumors showed a similar distribution of stages (FIG. 2A). 44% of control and mutant tumors were hyperplasia or adenoma while 33% of control and 44% of mutant were early carcinomas. Only 22 and 11% of control and mutant tumors had progressed to late carcinomas at this time point. At 22 weeks, about 90% of the control tumors were carcinomas and of these, the early-late carcinoma distribution was 30% and 60%. By contrast, the stromal Wnt7b deletion resulted in only 40% carcinomas with a equal distribution of early and late. Similarly, 12% of control tumors were hyperplasias or adenomas, but 56% of mutant tumors were at the same stage. Fisher's Exact Test showed that while the control tumors showed significant tumor progression between 16 and 22 weeks (p=0.05), the mutant tumors did not. These results show that Wnt7b from the hematopoietic tumor stroma promotes malignant tumor progression.

An important component of tumor progression is the establishment of a high density vasculature—the so-called angiogenic switch (Hanahan and Weinberg, 2011). To determine whether there was any indication of changed vascular density in mutant tumors, we used a flow cytometry protocol was used to count CD31+, CD 105+ vascular endothelial cells (VECs). This showed that at 22 weeks, there were reduced numbers of VECs in mutant tumors (FIG. 2B). To assess the density of functional vessels, tumor bearing mice were perfused with TEXAS-RED conjugated, lysine-fixable dextran and compared tumor stage with either vessels per field (FIG. 2C) or branch-points per field (FIG. 2E). At premalignant tumor stages, both control and mutant tumors displayed the low vascular density typical of tumors that have not undergone the angiogenic switch. In early and late carcinomas of control mice, the vascular density was greatly increased indicative of the angiogenic switch. By contrast, mutant mice showed no significant change in the vascular density even though some tumors had progressed to early and late carcinomas (FIG. 2C, 2E). To assess vascular changes using another marker, labeling was performed with CD31 in 22 week control and mutant tumors. Quantification of CD31+ area (FIG. 2D) and CD31+ vessels (FIG. 2F) showed that by this measure too, mutant tumors showed reduced vascular density. These data show that Wnt7b from the hematopoietic tumor stroma is required for the angiogenic switch and that this correlates with a reduced frequency of malignant tumors.

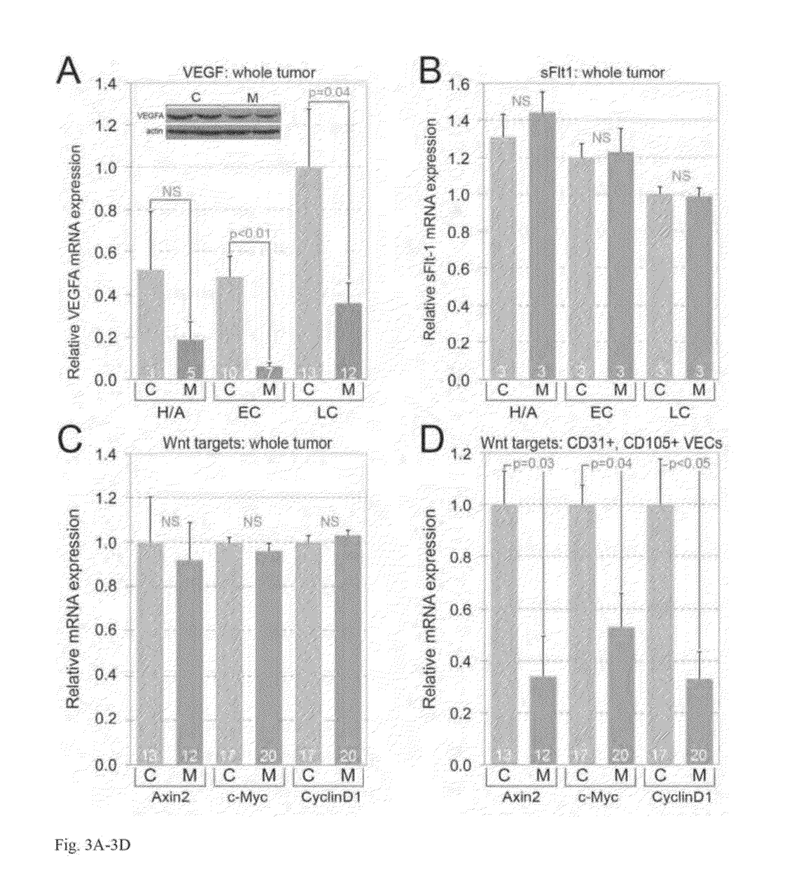

VEGFA is a key stimulator of angiogenesis and a target gene of the Wnt/β-catenin pathway in some tumors (Zhang et al., 2010). Therefore, it was assessed whether the failure of the angiogenic switch in mutant tumors might be associated with changes in VEGFA expression. In accordance with this hypothesis, the level of the VEGFA mRNA was significantly reduced in mutant tumors at both the early and late carcinoma stages (FIG. 3A). Moreover, this was mirrored by a discernibly reduced level of VEGFA protein according to whole-tumor Western blot (FIG. 3A, inset). FM is a naturally-occurring inhibitor of VEGFA activity (Kendall and Thomas, 1993) and it was possible that increased expression level could partly account for angiogenic switch failure in Wnt7b mutant tumors. However, QPCR assessment of Flt1 transcript levels in control and mutant tumors of all stages did not reveal any significant changes (FIG. 3B). These data support the suggestion that Wnt7b is critical in up-regulating expression of VEGFA that mediates angiogenic switching.

VECs are known to express the response machinery for the Wnt/β-catenin pathway (Goodwin and D'Amore, 2002; Lobov et al., 2005) and so it was possible that Wnt7b-dependent tumor progression was in part mediated by a direct effect. To assess the status of the Wnt/β-catenin pathway in tumor VECs, we performed rapid flow-sorting of CD31+, CD105+ cells from 22 week tumors and performed QPCR for Axin2, c-myc and CyclinD1, three established Wnt/3-catenin pathway target genes. As a control, we performed the same QPCR but from the whole tumor. This showed that while whole control and mutant tumors showed no significant changes (FIG. 3C) the three Wnt/β-catenin pathway target genes were all significantly reduced in VECs (FIG. 3D). These data identify VECs from the MMTV-PyMT tumor as a Wnt/β-catenin pathway responsive cell.

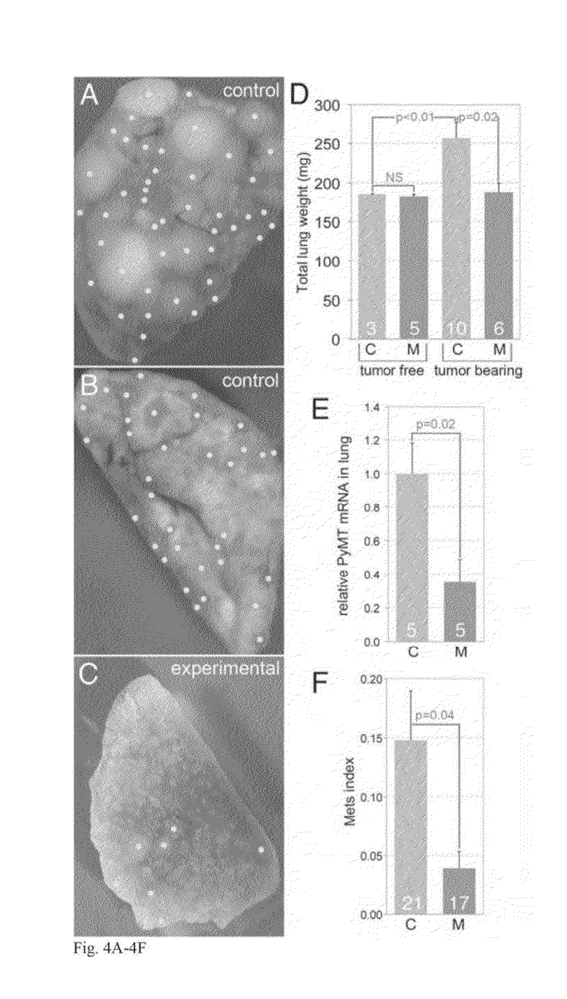

The favored sites of metastasis in the MMTV-PyMT tumor model are lung and lymph node (Lin et al., 2003). Lungs from tumor-bearing 22 week old control mice showed metastases over a range of severity represented by the images shown (FIGS. 4A and B). To quantify the rate of metastasis in the control and Wnt7b conditional mutant (FIG. 4C), the lungs of 22 week old mice were isolated and weighed. The lungs of non-tumor bearing mice served as a negative control. Control, MMTV-PyMT; Wnt7b^{-/tm2Amc} lungs were physically larger and weighed significantly more (FIG. 4D) than their control counterparts. Conditional deletion of Wnt7b returned the lung weight of tumor bearing mice to the control level (FIG. 4D). Using QPCR amplification of the MMTV-PyMT transcript as an alternative measure of lung metastatic load also showed that mutant mice had reduced metastasis (FIG. 4E). A metastatic index calculated according to established methods (Qian et al., 2009) showed, like lung weight and PyMT transcript, that loss of Wnt7b from the hematopoietic tumor stroma reduced the level of metastasis (FIG. 4F). These data show that Wnt7b expression in hematopoietic cells has an important role in promoting metastasis.

Herein it is thus shown that the Wnt family ligand Wnt7b has a key role in malignant progression of the MMTV-PyMT model of mammary carcinoma. Using conditional inactivation of the Wnt7b gene using Csflr-icre (Deng et al., 2010), it is shown that the critical source of Wnt7b is the hematopoietic tumor stroma. A key finding is that Wnt7b inactivation in the hematopoietic stroma results in reduced pulmonary metastasis. This is not likely to result from the inactivation of Wnt7b in B-cells as the complete absence of B-cells in the MMTV-PyMT tumor does not change the rate of pulmonary metastasis (DeNardo et al., 2009). However, it has recently been shown, using T-cell deficient mice, that CD4+ T-cells promote pulmonary metastasis. The evidence further suggests that T-cells influence the function of TAMs through the production of IL-4 (DeNardo et al., 2009). This may suggest a signaling relationship between the Wnt and IL-4 pathways or alternatively, that each pathway independently supports a response required for pulmonary metastasis, perhaps at different stages of the process.

Tumor stromal cells contribute to tumor progression in part by promoting angiogenesis (Lin et al., 2006). The absence of stromal B-cells and T-cells does not have an effect on stromal-dependent angiogenesis (DeNardo et al., 2009), and so despite the expression and inactivation of Wnt7b in lymphoid cells in this study, the likely Wnt7b-dependent mediator of the angiogenic switch is the TAM. This is consistent with prior work showing that macrophages can promote angiogenic switching (Lin et al., 2006) and with analysis showing that Wnt7b can regulate angiogenesis and vascular remodeling in other settings including the developing neuroepithelium (Stenman et al., 2008), the lung (Rajagopal et al., 2008; Shu et al., 2002) and via macrophages, in the eye (Lobov et al., 2005). The observation that conditional Wnt7b inactivation results in reduced expression of several canonical Wnt pathway target genes in VECs also suggests that cells in the hematopoietic stroma are likely to directly stimulate the vasculature with Wnt7b. Since Wnt7b has a short range-of-action (Lobov et al., 2005), it is likely that signaling is via cell-cell contact, a possibility consistent with the distribution of tumor stromal cells immediately adjacent to tumor vasculature.

VEGF is an established mediator of tumor angiogenesis (Ferrara et al., 2004). The current analysis shows that high VEGFA transcript and protein levels in the MMTV-PyMT tumor are dependent on stromal Wnt7b. Deletion of VEGFA from the myeloid tumor stroma using LysM-cre resulted in increased tumor size, the loss of angiogenic switching, no change in overall VEGFA expression but reduced VEGFA responsiveness (Stockmann et al., 2008). This contrasts with the current findings where conditional Wnt7b inactivation results in reduced tumor size, a globally reduced level of VEGFA expression, and a loss of angiogenic switching. These differences probably reflect a function for Wnt7b in directly eliciting responses from cells within the tumor and the likelihood that Wnt7b-induced VEGFA expression is just one of these responses. Indeed, the major effect of conditional deletion of Wnt7b on MMTV-PyMT tumor progression is very similar to that observed when macrophages are depleted or ablated (Lin et al., 2007; Lin et al., 2006; Qian et al., 2009). The role of Wnt7b in promoting many different aspects of tumor progression (three of the six recognized cancer hallmarks (Hanahan and Weinberg, 2011) are significantly reduced) shows therapeutic targeting of this pathway will be of benefit in the treatment of solid tumors and is expected to reduce the rate of metastasis, a key factor in patient survival.

### Methods and Materials

Mouse strains and Genotyping:

All experiments were conducted in accordance with guidelines of Institutional Animal Care and Use Committee. Mouse lines used in this study include the Wnt7b^{tm2Amc}conditional allele (Lin et al., 2010) and the MMTV-PyMT (Lin et al., 2003) and Csflr-icre (Deng et al., 2010) transgenic lines. The Wnt7b^{tm2Amc} allele was crossed with Ella-cre (Lakso et al., 1996) to generate Wnt7bgermline null. Genotyping was performed with primers: PyMT (Forward: 5′ GGAAGCAAGTACTTCACAAGGG 3′ (SEQ ID NO:3), Reverse: 5′ GGAAAGTCACTAGGAGCAGGG 3′, (SEQ ID NO:4)), Csf1riCre (Forward: 5′ CAGGGCCTTCTCCACACCAGC 3′ (SEQ ID NO:5), Reverse: 5′ CTGGCTGTGAAGACCATC 3′, (SEQ ID NO:6)), Wnt7b^{tm2Amc} (Forward 5′-TGACAGAGGATGGGGAGAAG-3′ (SEQ ID NO:7), Reverse: 5′-GGTCTTTCCAAGGGTGGTCT-3′) (SEQ ID NO:8), Wnt7b− (Forward: 5′-TATCCCACCGATACGCAAAC-3′ (SEQ ID NO:9), Reverse: 5′-GAGGAAGTCAGGCAGGTGTC-3′) (SEQ ID NO:10), Ella-cre (Forward: 5′-CCATTTGTGGCTGGTAACTC-3′ (SEQ ID NO:11), Reverse: 5′-CATGTTTAGCTGGCCCAAAT-3′ (SEQ ID NO:12)).

Flow Cytometry

Tumor-associated hematopoietic stromal and vascular endothelial cells were isolated by flow cytometry using labeling for anti-CD3-PE (Clone 17A2), anti-B220-APC (Clone RA3-6B2), ntimouse CD45-PE-Cy7 (Clone 30-F11), anti-F4/80-APC-eFluor 780 (Clone BM8), CD45 microbeads, anti-mouse CD31-APC (Clone 390), and anti-mouse CD105-PE (Clone MJ7/18). To increase isolation efficiency, macrophages were also labeled with fluorescent dextran via phagocytic uptake as previously described (Wyckoff et al, 2004).

Assessment of Wnt7b^{tm2Amc} deletion:

Flow sorted, tumor associated F4/80-positive macrophages, T cells, B cells and blood vessel endothelial cells were used for isolation of genomic DNA. The primers used to assess deletion of Wnt7b^{tm2Amc} were; forward: 5′-CCCAAGCGCTACCTTTCTG-3′ (SEQ ID NO:13), reverse: 5′-GCTACGGCATCGACTTTTCT-3′ (SEQ ID NO:14). Control actin primers were; forward 5′-CGGTGCTAAGAAGGCTGTTC-3′ (SEQ ID NO:15), reverse 5′-CTTCTCCATGTCGTCCCAGT-3′ (SEQ ID NO:16).

Histological analysis and volume quantification of mammary tumors The inguinal gland was removed, fixed in 4% paraformaldehyde, processed, sectioned and stained according to established procedures. For immunostaining, sections were deparaffinized, rehydrated and labeled using the TSA Detection kit (Invitrogen) and goat anti-PECAM antibody at 1:100 dilution (M-20, Santa Cruz). Functional blood vessels were identified using injected TEXAS RED-conjugated Dextran and their density quantified according to established procedures (Lin et al., 2006). Gland volume was calculated as described (Yeo et al., 2003).

RNA isolation and QPCR:

Lungs or whole tumors were ground to a fine powder in liquid nitrogen and subsequently homogenized. RNA was extracted from those tissues with TRIzol (Invitrogen) and used for quantitative PCR according to established procedures. The primers used were; VEGFA, forward 5′-GACAGAACAAAGCCAGA-3′ (SEQ ID NO:17), reverse: CACCGCCTTGGCTTGTCAC-3′ (SEQ ID NO:18), β-actin, forward: 5% TTCTTTGCAGCTCCTTCGTT-3′ (SEQ ID NO:19), reverse: 5′-ATGGAGGGGAATACAGCCC-3′ (SEQ ID NO:20), Flt1, forward, 5′-GGCATCCCTCGGCCAACAATC-3′ (SEQ ID NO:21), reverse, 5′-AGTTGCTGCTGGGATCCAGG-3′ (SEQ ID NO:22).

Assessment of the pulmonary metastatic burden was performed using QPCR for the PyMT transcript. Lungs were removed before removing mammary glands to avoid cross contamination. The PyMT primers used were; forward: 5′-CTCCAACAGATACACCCGCACATACT-3′ (SEQ ID NO:23), reverse: 5′-GCTGGTCTTGGTCGCTTTCTGGATAC-3′ (SEQ ID NO:24).

Immunoblotting:

Tumor samples were solubilized in lysis buffer consisting of 20 mM Tris (pH 7.5), 150 mM NaCl, 1 mM EDTA, 2% Triton X-100, 2.5 mM Sodium Pyrophosphate, 1 mM β-glycerophosphate, 1 mM Na₃VO4, protease inhibitor cocktail and 1 mM PMSF. 50 μg of lysate was used for immunoblotting. The antibodies used were rabbit anti-VEGF (A-20, Santa Cruz) and rabbit anti-β-tubulin (ab6046, Abcam).

Statistical analysis:

All statistical analyses were performed using SPSS for Windows version 18.0. The Student's t test and Mann-Whitney U test were used. For the frequency of tumor stage, Fisher's exact test was employed. A p value less than or equal to 0.05 was considered statistically significant.

### REFERENCES

Andreu, P., Johansson, M., Affara, N. I., Pucci, F., Tan, T., Junankar, S., Korets, L., Lam, J., Tawfik, D., DeNardo, D. G., et al. (2010). FcRgamma activation regulates inflammation associated squamous carcinogenesis. Cancer Cell 17, 121-134.
Bailey, A. S., Willenbring, H., Jiang, S., Anderson, D. A., Schroeder, D. A., Wong, M. H., Grompe, M., and Fleming, W. H. (2006). Myeloid lineage progenitors give rise to vascular endothelium. Proc Natl Acad Sci USA 103, 13156-13161.
Bui, T. D., Zhang, L., Rees, M. C., Bicknell, R., and Hanis, A. L. (1997). Expression and hormone regulation of Wnt2, 3, 4, 5a, 7a, 7b and 10b in normal human endometrium and endometrial carcinoma. Br J Cancer 75, 1131-1136.
Condeelis, J., and Pollard, J. W. (2006). Macrophages: obligate partners for tumor cell migration, invasion, and metastasis. Cell 124, 263-266.
Dahmen, R. P., Koch, A., Denkhaus, D., Tonn, J. C., Sorensen, N., Berthold, F., Behrens, J., Birchmeier, W., Wiestler, O. D., and Pietsch, T. (2001). Deletions of AXIN1, a component of the WNT/wingless pathway, in sporadic medulloblastomas. Cancer Res 61, 7039-7043.
De Palma, M., Venneri, M. A., Galli, R., Sergi, L. S., Politi, L. S., Sampaolesi, M., and Naldini, L. (2005). Tie2 identifies a hematopoietic lineage of proangiogenic monocytes required for tumor vessel formation and a mesenchymal population of pericyte progenitors. Cancer cell 8, 211-226.
de Visser, K. E., Korets, L. V., and Coussens, L. M. (2005). De novo carcinogenesis promoted by chronic inflammation is B lymphocyte dependent. Cancer Cell 7, 411-423.
DeNardo, D. G., Barreto, J. B., Andreu, P., Vasquez, L., Tawfik, D., Kolhatkar, N., and Coussens, L. M. (2009). CD4(+) T cells regulate pulmonary metastasis of mammary carcinomas by enhancing protumor properties of macrophages. Cancer Cell 16, 91-102.
Deng, L., Zhou, J. F., Sellers, R. S., Li, J. F., Nguyen, A. V., Wang, Y., Orlofsky, A., Liu, Q., Hume, D. A., Pollard, J. W., et al. (2010). A novel mouse model of inflammatory bowel disease links mammalian target of rapamycin-dependent hyperproliferation of colonic epithelium to inflammationassociated tumorigenesis. Am J Pathol 176, 952-967.
Ferrara, N., Hillan, K. J., Gerber, H. P., and Novotny, W. (2004). Discovery and development of bevacizumab, an anti-VEGF antibody for treating cancer. Nat Rev Drug Discov 3, 391-400.
Garcia-Rostan, G., Camp, R L., Herrero, A., Carcangiu, M. L., Rimm, D. L., and Tallini, G. (2001). Beta-catenin dysregulation in thyroid neoplasms: down-regulation, aberrant nuclear expression, and CTNNB1 exon 3 mutations are markers for aggressive tumor phenotypes and poor prognosis. Am J Pathol 158, 987-996.
Goodwin, A. M., and D'Amore, P. A. (2002). Wnt signaling in the vasculature. Angiogenesis 5, 1-9.
Hanahan, D., and Folkman, J. (1996). Patterns and emerging mechanisms of the angiogenic switch during tumorigenesis. Cell 86, 353-364.
Huguet, E. L., McMahon, J. A., McMahon, A. P., Bicknell, R., and Harris, A. L. (1994). Differential expression of human Wnt genes 2, 3, 4, and 7B in human breast cell lines and normal and disease states of human breast tissue. Cancer Res 54, 2615-2621.
Kendall, R. L., and Thomas, K. A. (1993). Inhibition of vascular endothelial cell growth factor activity by an endogenously encoded soluble receptor. Proc Natl Acad Sci USA 90, 10705-10709.
Kinzler, K. W., Nilbert, M. C., Su, L. K., Vogelstein, B., Bryan, T. M., Levy, D. B., Smith, K. J., Preisinger, A. C., Hedge, P., McKechnie, D., et al. (1991). Identification of FAP locus genes from chromosome 5q21. Science 253, 661-665.
Kinzler, K. W., and Vogelstein, B. (1996). Lessons from hereditary colorectal cancer. Cell 87, 159-170.
Korinek, V., Barker, N., Morin, P. J., van Wichen, D., de Weger, R., Kinzler, K. W., Vogelstein, B., and Clevers, H. (1997). Constitutive transcriptional activation by a beta-catenin-Tcf complex in APC−/− colon carcinoma. Science 275, 1784-1787.
Lakso, M., Pichel, J. G., Gorman, J. R., Sauer, B., Okamoto, Y., Lee, E., Alt, F. W., and Westphal, H. (1996). Efficient in vivo manipulation of mouse genomic sequences at the zygote stage. Proc Natl Acad Sci USA 93, 5860-5865.
Lin, E. Y., Jones, J. G., Li, P., au, L., Whitney, K. D., Muller, W. J., and Pollard, J. W. (2003). Progression to malignancy in the polyoma middle T oncoprotein mouse breast cancer model provides a reliable model for human diseases. Am J Pathol 163, 2113-2126.
Lin, E. Y., Li, J. F., Bricard, G., Wang, W., Deng, Y., Sellers, R., Porcelli, S. A., and Pollard, J. W. (2007). Vascular endothelial growth factor restores delayed tumor progression in tumors depleted of macrophages. Mol Oncol 1, 288-302.
Lin, E. Y., Li, J. F., Gnatovskiy, L., Deng, Y., Zhu, L., Grzesik, D. A., Qian, B., Xue, X. N., and Pollard, J. W. (2006). Macrophages regulate the angiogenic switch in a mouse model of breast cancer. Cancer research 66, 11238-11246.
Lin, E. Y., Nguyen, A. V., Russell, R. G., and Pollard, J. W. (2001). Colony-stimulating factor 1 promotes progression of mammary tumors to malignancy. J Exp Med 193, 727-740.
Lin, S. L., Li, B., Rao, S., Yeo, E. J., Hudson, T. E., Nowlin, B. T., Pei, H., Chen, L., Zheng, J. J., Carroll, T. J., et al. (2010). Macrophage Wnt7b is critical for kidney repair and regeneration. Proc Natl Acad Sci USA 107, 4194-4199.
Lindvall, C., Evans, N. C., Zylstra, C. R., Li, Y., Alexander, C. M., and Williams, B. O. (2006). The Wnt signaling receptor Lrp5 is required for mammary ductal stem cell activity and Wnt1-induced tumorigenesis. J Biol Chem 281, 35081-35087.
Lobov, L B., Rao, S., Carroll, T. J., Valiance, J. E., Ito, M., Ondr, J. K., Kurup, S., Glass, D. A., Patel, M. S., Shu, W., et al. (2005). WNT7b mediates macrophage-induced programmed cell death in patterning of the vasculature. Nature 437, 417-421.
Logan, C. Y., and Nusse, R. (2004). The Wnt signaling pathway in development and disease. Annu Rev Cell Dev Biol 20, 781-810.
Matsui, S., Ahlers, J. D., Vortmeyer, A. O., Terabe, M., Tsukui, T., Carbone, D. P., Liotta, L. A., and Berzofsky, J. A. (1999). A model for CD8+CTL tumor immunosurveillance and regulation of tumor escape by CD4 T cells through an effect on quality of CTL. J Immunol 163, 184-193.
Nusse, R, and Varmus, H. E. (1982). Many tumors induced by the mouse mammary tumor virus contain a provirus integrated in the same region of the host genome. Cell 31, 99-109.
Ojalvo, L. S., King, W., Cox, D., and Pollard, J. W. (2009a). High-density gene expression analysis of tumor-associated macrophages from mouse mammary tumors. Am J Pathol 174, 1048-1064.
Ojalvo, L. S., Whittaker, C. A., Condeelis, J. S., and Pollard, J. W. (2009b). Gene expression analysis of macrophages that facilitate tumor invasion supports a role for Wnt-signaling in mediating their activity in primary mammary tumors. J Immunol 184, 702-712.
Ojalvo, L. S., Whittaker, C. A., Condeelis, J. S., and Pollard, J. W. (2010). Gene expression analysis of macrophages that facilitate tumor invasion supports a role for Wnt-signaling in mediating their activity in primary mammary tumors. J Immunol 184, 702-712.
Pukkila, M. J., Virtaniemi, J. A., Kumpulainen, E. J., Pirinen, R. T., Johansson, R. T., Valtonen, H. J., Juhola, M. T., and Kosma, V. M. (2001). Nuclear beta catenin expression is related to unfavourable outcome in oropharyngeal and hypopharyngeal squamous cell carcinoma. J Clin Pathol 54, 42-47.
Qian, B., Deng, Y., Im, J. H., Muschel, R. J., Zou, Y., Li, J., Lang, R. A., and Pollard, J. W. (2009). A distinct macrophage population mediates metastatic breast cancer cell extravasation, establishment and growth. PLoS ONE 4, e6562.
Rajagopal, J., Carroll, T. J., Guseh, J. S., Bores, S. A., Blank, L. J., Anderson, W. J., Yu, J., Zhou, Q., McMahon, A. P., and Melton, D. A. (2008). Wnt7b stimulates embryonic lung growth by coordinately increasing the replication of epithelium and mesenchyme. Development 135, 1625-1634.
Shu, W., Jiang, Y. Q., Lu, M. M., and Morrisey, E. E. (2002). Wnt7b regulates mesenchymal proliferation and vascular development in the lung. Development 129, 4831-4842.
Stenman, J. M., Rajagopal, J., Carroll, T. J., Ishibashi, M., McMahon, J., and McMahon, A. P. (2008).
Canonical Wnt signaling regulates organ-specific assembly and differentiation of CNS vasculature. Science 322, 1247-1250.
Stockmann, C., Doedens, A., Weidemann, A., Zhang, N., Takeda, N., Greenberg, Cheresh, D. A., and Johnson, R. S. (2008). Deletion of vascular endothelial growth factor in myeloid cells accelerates tumorigenesis. Nature 456, 814-818.
Taniguchi, K., Roberts, L. R., Aderca, I. N., Dong, X., Qian, C., Murphy, L. M., Nagorney, D. M., Burgart, L. J., Roche, P. C., Smith, D. I., et al. (2002). Mutational spectrum of beta-catenin, AXIN1, and AXIN2 in hepatocellular carcinomas and hepatoblastomas. Oncogene 21, 4863-4871.
Wang, L., Yi, T., Kortylewski, M., Pardoll, D. M., Zeng, D., and Yu, H. (2009). IL-17 can promote tumor growth through an IL-6-Stat3 signaling pathway. J Exp Med 206, 1457-1464.
Wyckoff, J., Wang, W., Lin, E. Y., Wang, Y., Pixley, F., Stanley, E. R., Graf, T., Pollard, J. W., Segall, J., and Condeelis, J. (2004). A paracrine loop between tumor cells and macrophages is required for tumor cell migration in mammary tumors. Cancer Res 64, 7022-7029.
Yeo, E. J., Chun, Y. S., Cho, Y. S., Kim, J., Lee, J. C., Kim, M. S., and Park, J. W. (2003). YC-1: a potential anticancer drug targeting hypoxia-inducible factor 1. J Natl Cancer Inst 95, 516-525.
Zhang, B., Abreu, J. G., Zhou, K., Chen, Y., Hu, Y., Zhou, T., He, X., and Ma, J. X. (2010). Blocking the Wnt pathway, a unifying mechanism for an angiogenic inhibitor in the serine proteinase inhibitor family. Proc Natl Acad Sci USA 107, 6900-6905.

1. A method of inhibiting formation of blood vessels in a malignant tumor in a subject, or of inhibiting metastasis of a malignant tumor in a subject, comprising administering to the subject an inhibitor of Wnt7b in an amount effective to inhibit formation of blood vessels in a malignant tumor or metastasis of the malignant tumor in the subject, respectively.

2. The method of claim 1, wherein the inhibitor of Wnt7b is an inhibitor of macrophage-secreted Wnt7b or hematopoietic stroma-derived Wnt7b.

3. The method of claim 1, wherein the inhibitor of Wnt7b inhibits angiogenesis or vasculogenesis in the malignant tumor.

4. The method of claim 1, wherein inhibitor of Wnt7b inhibits metastasis of the malignant tumor.

5. The method of claim 1, wherein the malignant tumor is a tumor of the breast, prostate, lung, liver, cervix, pancreas, kidney, ovary, testicle, uterus, glia, central nervous system, oesphagus, stomach, colon, or is a glioblastoma, or wherein the metastasis is a pulmonary, bone, liver, breast, melanoma, kidney, cervix, or colon metastasis.

6. The method of claim 1, wherein the inhibitor of Wnt7b is an antibody which binds Wnt7b or a fragment of an antibody which fragment binds Wnt7b.

7. The method of claim 6, wherein the inhibitor of Wnt7b is a monoclonal antibody or a fragment of a monoclonal antibody.

8. (canceled)

9. The method of claim 1, wherein the inhibitor of Wnt7b is an antisense which inhibits expression of Wnt7b.

10. The method of claim 1, wherein the inhibitor of Wnt7b is an siRNA which inhibits expression of Wnt7b or an shRNA which inhibits expression of Wnt7b.

11. The method of claim 1, wherein the inhibitor of Wnt7b is a small organic molecule of 2000 daltons or less.

12-13. (canceled)

14. A method of inhibiting a benign tumor from transforming into a malignant tumor in a subject comprising administering to the subject an inhibitor of Wnt7b in an amount effective to inhibit a benign tumor from transforming into a malignant tumor.

15. The method of claim 14, wherein the inhibitor of Wnt7b is an inhibitor of macrophage-secreted Wnt7b.

16. (canceled)

17. The method of claim 14, wherein the inhibitor of Wnt7b is an antibody which binds Wnt7b or a fragment of an antibody which fragment binds Wnt7b.

18. The method of claim 17, wherein the inhibitor of Wnt7b is a monoclonal antibody or a fragment of a monoclonal antibody.

19. (canceled)

20. The method of claim 14, wherein the inhibitor of Wnt7b is an antisense which inhibits expression of Wnt7b.

21. The method of claim 14, wherein the inhibitor of Wnt7b is an siRNA which inhibits expression of Wnt7b or an shRNA which inhibits expression of Wnt7b.

22. The method of claim 14, wherein the inhibitor of Wnt7b is a small organic molecule of 2000 daltons or less.

23. The method of claim 22, wherein the small organic molecule is pyrvinium(2-[(E)-2-(2,5-Dimethyl-1-phenylpyrrol-3-yl)ethenyl]-N,N,1-trimethylquinolin-1-ium-6-amine).

24. The method of claim 14, wherein the inhibitor is a secreted frizzled protein or an active fragment thereof.

25. A method for identifying an agent as an inhibitor of angiogenesis and/or vasculogenesis in a malignant tumor or as an inhibitor of metastasis of a malignant tumor, comprising:
contacting an amount of Wnt7b with an amount of the agent and determining whether the agent inhibits the activity of the Wnt7b,
wherein if the agent inhibits the activity of the Wnt7b then the agent is an inhibitor of angiogenesis and/or vasculogenesis in a malignant tumor or an inhibitor of metastasis of a malignant tumor, and wherein if the agent does not inhibit the Wnt7b then the agent is not identified as an inhibitor of angiogenesis and/or vasculogenesis in a malignant tumor and/or an inhibitor of metastasis of a malignant tumor.

26-34. (canceled)


SEQUENCE LISTING (24 sequences)

SEQ ID NO: 1 - protein, 349 aa, HOMO SAPIENS
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 2 - DNA, 2157 bp, HOMO SAPIENS
ggatcatgcacagaaactttcgcaagtggattttctacgtgtttctctgctttggcgtcctgtacgtgaagctcggagcactgtcatccgtggtggccctgggagccaacatcatctgcaacaagattcctggcctagccccgcggcagcgtgccatctgccagagtcggcccgatgccatcattgtgattggggagggggcgcagatgggcatcaacgagtgccagtaccagttccgcttcggacgctggaactgctctgccctcggcgagaagaccgtcttcgggcaagagctccgagtagggagccgtgaggctgccttcacgtacgccatcaccgcggctggcgtggcgcacgccgtcaccgctgcctgcagccaagggaacctgagcaactgcggctgcgaccgcgagaagcagggctactacaaccaagccgagggctggaagtggggcggctgctcggccgacgtgcgttacggcatcgacttctcccggcgcttcgtggacgctcgggagatcaagaagaacgcgcggcgcctcatgaacctgcataacaatgaggccggcaggaaggttctagaggaccggatgcagctggagtgcaagtgccacggcgtgtctggctcctgcaccaccaaaacctgctggaccacgctgcccaagttccgagaggtgggccacctgctgaaggagaagtacaacgcggccgtgcaggtggaggtggtgcgggccagccgtctgcggcagcccaccttcctgcgcatcaaacagctgcgcagctatcagaagcccatggagacagacctggtgtacattgagaagtcgcccaactactgcgaggaggacgcggccacgggcagcgtgggcacgcagggccgtctctgcaaccgcacgtcgcccggcgcggacggctgtgacaccatgtgctgcggccgaggctacaacacccaccagtacaccaaggtgtggcagtgcaactgcaaattccactggtgctgcttcgtcaagtgcaacacctgcagcgagcgcaccgaggtcttcacctgcaagtgaggccaggcccggaggcggccgcgggcaccctggaacccggcggcattttgcacatccactcctcaccttccctgccttggtgctgccagcagcagacatagacgggtgcagaagcggggagctccaggtgcaggagggcaccggccggggcccacgccctctgcccgcctccctggggctccttcctgccacctcctcccatcacctcctgcggcagaacagcacccgtgacccacccagagagcaaggccaggggtcttggtgctccccgacggggcccggcaagttctctttcttctctctgggaaaatgaacgtccaggacacacctgtatcccagagagcaaagtgatgaggagactgagcgtccccagccccacctggcggcatggacacagaaaagctacgccggctggcctctccagaccagttcccaggctgggtctgctgggccctggggcggtggggacagatgttgacacaaattatttatgttttcttagtatcagaagaggattctcggcactaacacatagccagtcctaactccgtactctgtgtcagcccatcccctagacaccctctgtttcctttcccggccccacctggccggccctctgcccctgcagagctgaggcagcctggggttgatggggaccacgcggtgcctgcaggtcctagaagtgagctgggcaggggctcttcagaccacacagccctgaccgggccttggaggagagccatggacaggctcctccatgccgtctttccttcttttgaaaatcctatcaatggctgggcgcggtggctcacacctgtaatcccagcactttgggagaccgaggcaggtggatcacctgaggtcaggagttcgagaccagcctggccaacgtggtgaaaccctgtctctactaaaaatacaaaaattagctgggcgtggtggcgtgcacctgtaatcccagctactcaggaggctgagacaggacacttgcttgaacccgggaggtggaggttgcaatgagccaagattgtgccactgtattccaacttgggtgacagagcacgactctgtctcaaaaaaaaaaaaaaaaaaaaaaaaaaaa

SEQ ID NO: 3 - DNA, 22 bp, ARTIFICIAL SEQUENCE
  MOUSE PRIMER
ggaagcaagtacttcacaaggg

SEQ ID NO: 4 - DNA, 21 bp, ARTIFICIAL SEQUENCE
  MOUSE PRIMER
ggaaagtcactaggagcaggg

SEQ ID NO: 5 - DNA, 21 bp, ARTIFICIAL SEQUENCE
  MOUSE PRIMER
cagggccttctccacaccagc

SEQ ID NO: 6 - DNA, 18 bp, ARTIFICIAL SEQUENCE
  MOUSE PRIMER
ctggctgtgaagaccatc

SEQ ID NO: 7 - DNA, 20 bp, ARTIFICIAL SEQUENCE
  MOUSE PRIMER
tgacagaggatggggagaag

SEQ ID NO: 8 - DNA, 20 bp, ARTIFICIAL SEQUENCE
  MOUSE PRIMER
ggtctttccaagggtggtct

SEQ ID NO: 9 - DNA, 20 bp, ARTIFICIAL SEQUENCE
  MOUSE PRIMER
tatcccaccgatacgcaaac

SEQ ID NO: 10 - DNA, 20 bp, ARTIFICIAL SEQUENCE
  MOUSE PRIMER
gaggaagtcaggcaggtgtc

SEQ ID NO: 11 - DNA, 20 bp, ARTIFICIAL SEQUENCE
  MOUSE PRIMER
ccatttgtggctggtaactc

SEQ ID NO: 12 - DNA, 20 bp, ARTIFICIAL SEQUENCE
  MOUSE PRIMER
catgtttagctggcccaaat

SEQ ID NO: 13 - DNA, 19 bp, ARTIFICIAL SEQUENCE
  MOUSE PRIMER
cccaagcgctacctttctg

SEQ ID NO: 14 - DNA, 20 bp, ARTIFICIAL SEQUENCE
  MOUSE PRIMER
gctacggcatcgacttttct

SEQ ID NO: 15 - DNA, 20 bp, ARTIFICIAL SEQUENCE
  MOUSE PRIMER
cggtgctaagaaggctgttc

SEQ ID NO: 16 - DNA, 20 bp, ARTIFICIAL SEQUENCE
  MOUSE PRIMER
cttctccatgtcgtcccagt

SEQ ID NO: 17 - DNA, 17 bp, ARTIFICIAL SEQUENCE
  MOUSE PRIMER
gacagaacaaagccaga

SEQ ID NO: 18 - DNA, 19 bp, ARTIFICIAL SEQUENCE
  MOUSE PRIMER
caccgccttggcttgtcac

SEQ ID NO: 19 - DNA, 20 bp, ARTIFICIAL SEQUENCE
  MOUSE PRIMER
ttctttgcagctccttcgtt

SEQ ID NO: 20 - DNA, 19 bp, ARTIFICIAL SEQUENCE
  MOUSE PRIMER
atggaggggaatacagccc

SEQ ID NO: 21 - DNA, 21 bp, ARTIFICIAL SEQUENCE
  MOUSE PRIMER
ggcatccctcggccaacaatc

SEQ ID NO: 22 - DNA, 20 bp, ARTIFICIAL SEQUENCE
  MOUSE PRIMER
agttgctgctgggatccagg

SEQ ID NO: 23 - DNA, 26 bp, ARTIFICIAL SEQUENCE
  MOUSE PRIMER
ctccaacagatacacccgcacatact

SEQ ID NO: 24 - DNA, 26 bp, ARTIFICIAL SEQUENCE
  MOUSE PRIMER
gctggtcttggtcgctttctggatac